(19) United States
(12) Patent Application Publication
(10) Pub. No.: US 2025/0072769 A1
(43) Pub. Date: Mar. 6, 2025

(54) SENSOR MODULE FOR MEASURING BLOOD PRESSURE

(71) Applicant: CHARMCARE CO., LTD., Seoul (KR)

(72) Inventor: Dong Hwa LEE, Yongin-si (KR)

(21) Appl. No.: 18/518,352

(22) Filed: Nov. 22, 2023

Related U.S. Application Data
(63) Continuation of application No. PCT/KR2023/018023, filed on Nov. 10, 2023.

(30) Foreign Application Priority Data
Sep. 4, 2023 (KR) 10-2023-0116648

(51) Int. Cl. A61B 5/021

(52) U.S. Cl. CPC A61B 5/02141; A61B 5/742; A61B 2562/043

(57) ABSTRACT

A sensor module for measuring blood pressure includes a base portion, a pressure equalization sheet placed on one side of the base portion and filled with liquid or gas, a sensor unit including at least one sensor placed on one side of the pressure equalization sheet, and stoppers configured to be in close contact with the pressure equalization sheet and face each other.

### BACKGROUND

The present disclosure relates to a sensor module for measuring blood pressure.

Blood pressure is an indicator for interpreting circulatory diseases, and risk analysis is performed based on the blood pressure, which is effective in preventing cardiovascular diseases, such as stroke, heart failure, and myocardial infarction. Also, morning hypertension, in which blood pressure rises early in the morning, is related to heart disease and stroke, and in particular, it was found that the symptom of a sudden rise in blood pressure about 1 hour to 1.5 hours after waking up is causally related to stroke.

Therefore, understanding a correlation between time and blood pressure changes is useful in analyzing the risk of cardiovascular diseases, and accordingly, it is necessary to measure blood pressure continuously over a long period of time.

Accordingly, research is being actively conducted on wearable devices for measuring blood pressure that may easily check a user's blood pressure.

An example of the related art includes Korea Patent Application No. 10-2016-0063471 (Title of invention: WRISTBAND TYPE BLOOD PRESSURE MEASURING APPARATUS).

### SUMMARY

The present disclosure provides a sensor module that may accurately measure blood pressure by supporting a plurality of sensors with uniform pressure.

Objects to be solved by the present disclosure are not limited to the objects described above, and there may be other objects.

According to an aspect of the present disclosure, a sensor module for measuring blood pressure includes a base portion, a pressure equalization sheet placed on one side of the base portion and filled with liquid or gas, a sensor unit including at least one sensor placed on one side of the pressure equalization sheet, and stoppers configured to be in close contact with the pressure equalization sheet and face each other.

Also, heights of the stoppers may be lower than the pressure equalization sheet.

Also, the stoppers may surround the pressure equalization sheet.

Also, the sensor module may further include a circuit, wherein the circuit may include a communication unit configured to communicate with an external device, a controller configured to receive a sensor signal measured by the sensor unit, a display configured to display information received from the controller, and a battery configured to supply power to at least one of the communication unit, the controller, the display, and the sensor unit.

Also, the sensor unit may include a first sensor, a second sensor separated by a preset distance from the first sensor, and a step difference portion placed on one side of the first sensor, and a reference surface of the first sensor may have a step difference by a first distance with a reference surface of the second sensor.

Also, the sensor unit may further include a third sensor separated by a preset distance from the second sensor.

### BRIEF DESCRIPTION OF THE DRAWINGS

Embodiments of the inventive concept will be more clearly understood from the following detailed description taken in conjunction with the accompanying drawings in which:

FIG. 1 is a perspective view of a sensor module for measuring blood pressure, according to the first embodiment of the present disclosure;

FIG. 2 is a cross-sectional view of the sensor module for measuring the blood pressure, according to the first embodiment of the present disclosure;

FIG. 3 is a perspective view of a sensor module for measuring blood pressure, according to the second embodiment of the present disclosure;

FIG. 4 is a perspective view of a sensor module for measuring blood pressure according to the third embodiment of the present disclosure;

FIG. 5 is a perspective view of a sensor module for measuring blood pressure, according to the fourth embodiment of the present disclosure; and

FIG. 6 is a block diagram of a circuit according to an embodiment of the present disclosure.

### DETAILED DESCRIPTION OF THE EMBODIMENTS

Hereinafter, embodiments of the present disclosure will be described in detail such that those skilled in the art to which the present disclosure belongs may easily implement the present disclosure with reference to the accompanying drawings. However, the present disclosure may be implemented in many different forms and is not limited to the embodiments to be described herein. In addition, in order to clearly describe the present disclosure with reference to the drawings, portions irrelevant to the description are omitted, and similar reference numerals are attached to similar portions throughout the specification.

When it is described that a portion is “connected” to another portion throughout the specification, this includes not only a case where the portion is “directly connected” to another portion but also a case where the portion is “indirectly connected” to another portion with another component therebetween.

When it is described that a member is “on” another member throughout the specification, this includes not only a case where a member is in contact with another member, but also a case where there is another member between the two members.

Hereinafter, embodiments of the present disclosure are described in detail with reference to the attached drawings.

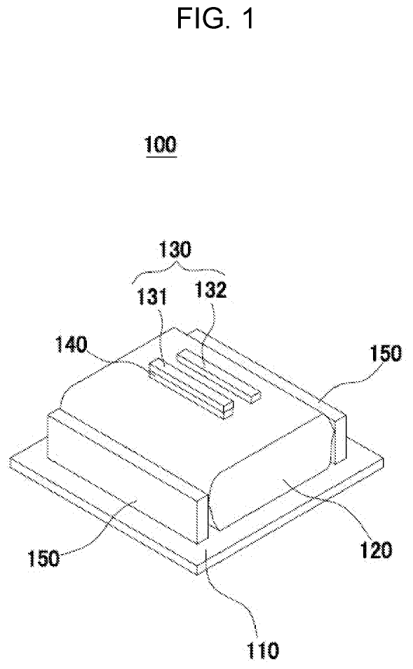

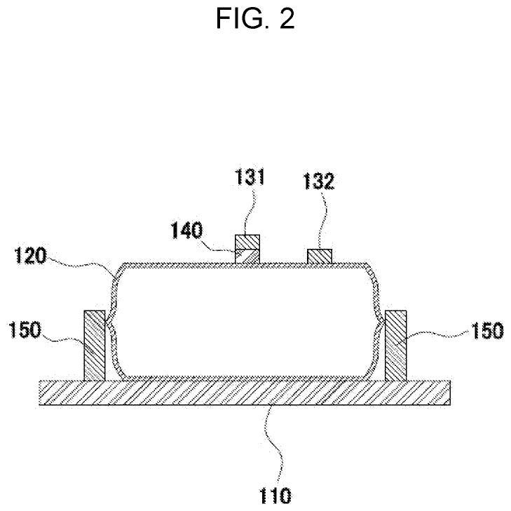

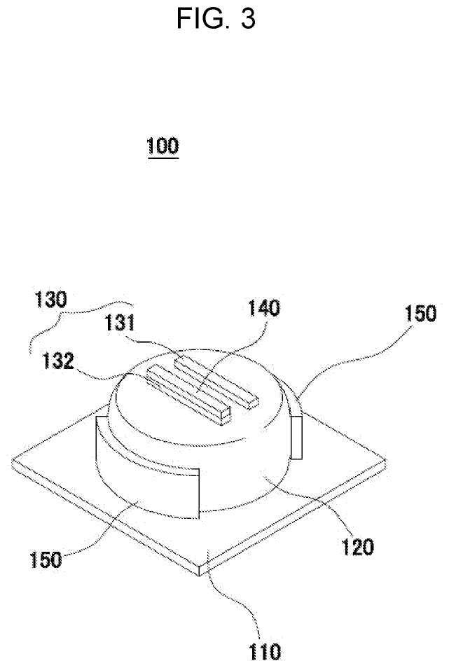

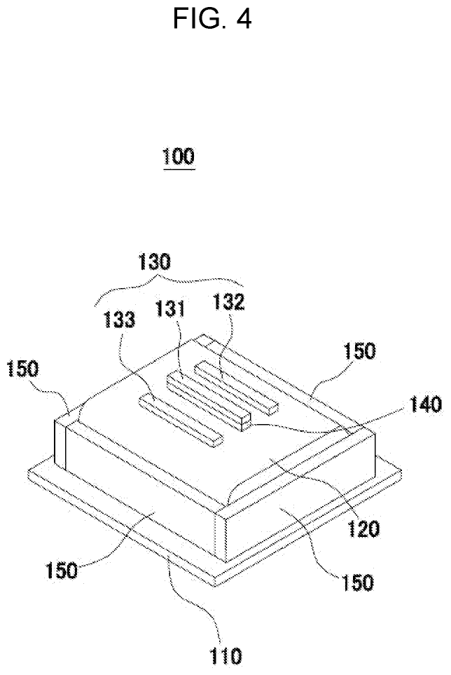

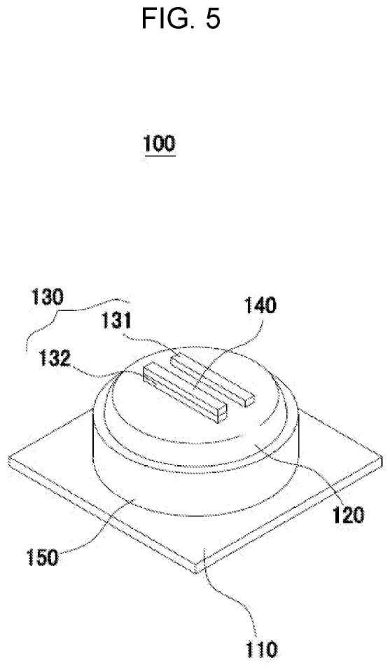

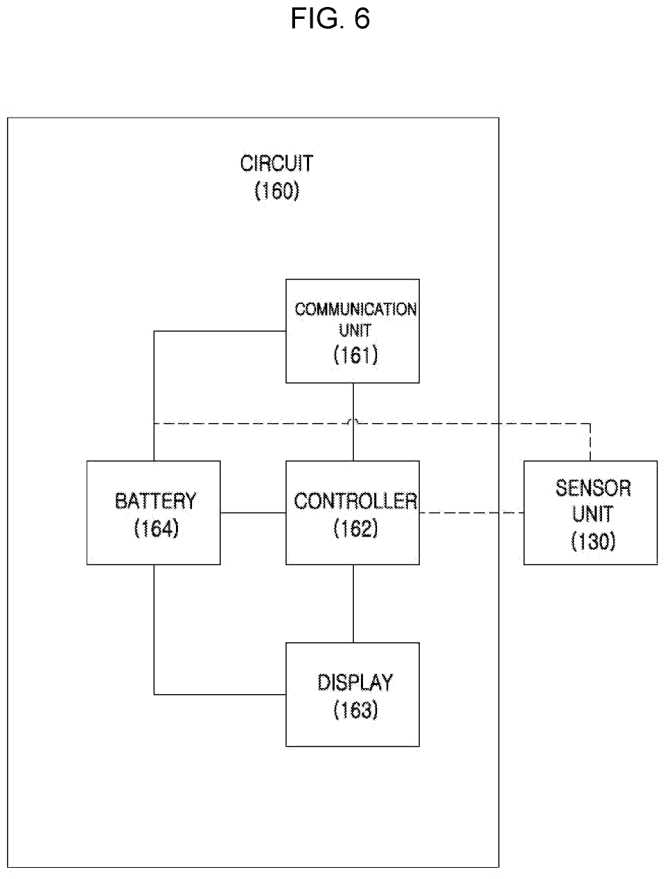

FIG. 1 is a perspective view of a sensor module 100 for measuring blood pressure, according to the first embodiment of the present disclosure, FIG. 2 is a cross-sectional view of the sensor module 100 for measuring the blood pressure, according to the first embodiment of the present disclosure, FIG. 3 is a perspective view of a sensor module 100 for measuring blood pressure, according to the second embodiment of the present disclosure, FIG. 4 is a perspective view of a sensor module 100 for measuring blood pressure according to the third embodiment of the present disclosure, FIG. 5 is a perspective view of a sensor module 100 for measuring blood pressure, according to the fourth embodiment of the present disclosure, and FIG. 6 is a block diagram of a circuit 160 according to an embodiment of the present disclosure.

The sensor module 100 for measuring blood pressure (hereinafter referred to as “sensor module 100”) according to an embodiment of the present disclosure includes a base portion 110, a pressure equalization sheet 120, a sensor unit 130, and stoppers 150. In addition, the sensor module 100 may be mounted on a wrist strap for measuring a user's blood pressure to measure a user's arterial blood pressure through the sensor unit 130 but is not limited thereto.

The base portion 110 may be formed in a plate shape with one side being flat. Also, the base portion 110 may support the pressure applied to the pressure equalization sheet 120 to be described below, and for this purpose, the base portion 110 may be formed of a material having a preset strength.

The pressure equalization sheet 120 is placed on one side of the base portion 110 and is filled with fluid, such as liquid or gas, therein. Also, the pressure equalization sheet 120 may cause the sensor unit 130 to come into close contact with a measurement target portion with a uniform pressure.

For example, the pressure equalization sheet 120 may be formed of a flexible material and may be provided with liquid or air filled therein but is not limited thereto and may be connected to a pump that provides liquid or air to provide liquid or air to the pressure equalization sheet 120 only when measuring blood pressure such that the inside of the pressure equalization sheet 120 maintains a constant pressure.

The sensor unit 130 may include at least one sensor provided on one side of the pressure equalization sheet 120.

Referring to FIG. 2, the sensor unit 130 may include a first sensor 131, a second sensor 132 separated from the first sensor 131 by a preset distance, and a step distance portion 140 placed on one side of the first sensor 131. Also, a reference surface of the first sensor 131 may have a step difference equal to a first distance with a reference surface of the second sensor 132.

For example, as illustrated in FIG. 2, the first sensor 131 may be placed on an upper portion of the step difference portion 140, and the step difference portion 140 and the second sensor 132 may be placed on the reference surface of the pressure equalization sheet 120. However, the present disclosure is not limited thereto, and as illustrated in FIG. 3, the first sensor 131 and the second sensor 132 may be placed on the reference surface of the pressure equalization sheet 120, and the step difference portion 140 may be placed on an upper portion of the first sensor unit 131. In this case, the first sensor 131 may measure blood pressure of a portion in contact with an upper surface of the first sensor 131, and the second sensor 132 may measure blood pressure of a portion in contact with an upper surface of the step difference portion 140. Accordingly, the first sensor 131 and the second sensor 132 each have a distance difference to the artery, and the sensor module 100 may measure blood pressure by using the distance difference. In this case, the step difference portion 140 may be integrated with the second sensor 132 or may be a separate component.

In this case, the first sensor 131 and the second sensor 132 may each be composed of any one of a strain gauge-based semiconductor thin film sensor, a capacitive thin film sensor that detects a change in capacitance according to pressure, a piezoresistive sensor using a piezoresistive effect, and a variety of other pressure sensors.

Here, the strain gauge-based thin film sensor has an advantage in which four resistors are arranged in a diaphragm in the form of a Wheatstone bridge and only a change in resistance is controlled. The capacitive thin film sensor has an advantage in which heat-resistance and corrosion-resistance are strong and pressure may be measured with high precision. The piezoresistive sensor has an advantage in which sensitivity is high and linearity and reproducibility are excellent when measuring pressure and mass production may be easily made.

The stoppers 150 may each be placed on one side of the base portion 110, be in close contact with the pressure equalization sheet 120, and face each other. In other words, the stoppers 150 may support the front and rear of the pressure equalization sheet 120, and when pressure is applied to the sensor unit 130 and the pressure is transferred to the pressure equalization sheet 120, the pressure equalization sheet 120 may is supported by the stoppers 150 to prevent a shape of the pressure equalization sheet 120 from being deformed. Accordingly, the pressure equalization sheet 120 may support the first sensor 131 and the second sensor 132 at a constant pressure, and thus, blood pressure may be stably measured by the first sensor 131 and the second sensor 132.

Also, when the sensor unit 130 comes into contact with a measurement target portion, upper surfaces of the stoppers 150 may be in contact with a surface of the measurement target portion. In addition, when no pressure is applied to the sensor unit 130, the upper surfaces of the stoppers 150 may be lower than measurement surfaces of the first sensor 131 and the second sensor 132. In other words, heights of the stoppers 150 may be lower than a height of the pressure equalization sheet 120.

also, as illustrated in FIG. 1, the stoppers 150 may each have a bar shape and face each other, and the pressure equalization sheet 120 may be placed between the stoppers 150. However, the stoppers 150 are not limited thereto and may each have an arc shape as illustrated in FIG. 3 and face each other.

Also, referring to FIGS. 4 and 5, the stoppers 150 may surround the pressure equalization sheet 120. In other words, the stoppers 150 may support the pressure equalization sheet 120 by being in close contact with a circumference of the pressure equalization sheet 120.

For example, referring to FIG. 4, the stoppers 150 may be formed on one side of the base portion 110 to have a preset height and each have a shape of a square pillar with an open top, and the pressure equalization sheet 120 may be inside the stoppers 150. Also, referring to FIG. 5, the stoppers 150 may be formed on one side of the base portion 110 to have a preset height and each have a cylindrical shape, and the pressure equalization sheet 120 may be placed inside the stoppers 150. In this case, the stoppers 150 may be formed into a single cylindrical shape by combining a plurality of stoppers 150 having an arc shape or may be formed as a single cylindrical member.

The pressure equalization sheet 120 is not deformed even when pressure is applied to the sensor unit 130 through the stoppers 150 on a circumference of the pressure equalization sheet 120 and the pressure is transferred to the pressure equalization sheet 120, and may stably supported the first sensor 131 and the second sensor 132 at a constant pressure.

Also, referring to FIG. 4, the sensor module 100 may further include a third sensor 133 placed at a preset distance apart from the second sensor 132. In other words, the sensor unit 130 may include three sensors placed at a preset distance apart from each other. However, the sensor unit 130 is not limited thereto and may include four or more sensors. Also, a controller 162 may be used to measure pressure by using a plurality of sensors and compare the measured values to calculate an optimal value or determine whether the plurality of sensors are abnormal.

Hereinafter, the circuit 160 according to the embodiment of the present disclosure is described with reference to FIG. 6.

The sensor module 100 may further include the circuit 160. The circuit 160 may include a communication unit 161, the controller 162, a display 163, and a battery 164.

The communication unit 161 may communicate with an external device. In detail, the communication unit 161 may communicate with various external devices (servers or terminals) in a set communication format to transmit and receive data.

The controller 162 may receive a sensor signal measured by the sensor unit 130. Also, the controller 162 may control the sensor unit 130 to measure blood pressure through the communication unit 161 when blood pressure measurement is needed and display the measured information on the display 163. For example, blood pressure may be measured on an hourly basis, the measured information may be displayed on the display 163, and when the measured blood pressure is out of a preset range, an alarm may be generated or information may be transferred to a designated user through the communication unit 161.

The display 163 may display the information received from the controller 162. By way of example, the display 163 may be implemented as various types of display monitors, such as a liquid crystal display, a reflective display, and an organic light emitting diode (OLED) display. The display 163 may display blood pressure or other types of information calculated by the controller 162.

The battery 164 may supply power to at least one of the communication unit 161, the controller 162, the display 163, and the sensor unit 130.

According to the present disclosure, a sensor module for measuring blood pressure according to an embodiment of the present disclosure may support a plurality of sensors to come into close contact with a measurement target region with a uniform pressure, and thus, blood pressure may be accurately measured.

One embodiment of the present disclosure may be implemented in the form of a recording medium including instructions executable by a computer, such as a program module executed by a computer. A computer readable medium may be any available medium that may be accessed by a computer and includes both volatile and nonvolatile media, removable and non-removable media. Also, the computer readable medium may include a computer storage medium. A computer storage medium includes both volatile and nonvolatile media and removable and non-removable media implemented by any method or technology for storing information, such as computer readable instructions, data structures, program modules or other data.

Although the method and systems of the present disclosure are described with reference to specific embodiments, some or all of their components or operations may be implemented by using a computer system having a general-purpose hardware architecture.

The above description of the present disclosure is for illustrative purposes, and those skilled in the art to which the present disclosure belongs will understand that the present disclosure may be easily modified into another specific form without changing the technical idea or essential features of the present disclosure. Therefore, the embodiments described above should be understood as illustrative in all respects and not limiting. For example, each component described as a single type may be implemented in a distributed manner, and likewise, components described as distributed may be implemented in a combined form.

The scope of the present disclosure is indicated by the following claims rather than the detailed description above, and the meaning and scope of the claims and all changes or modifications derived from the equivalent concepts should be interpreted as being included in the scope of the present disclosure.

What is claimed is:

1. A sensor module for measuring blood pressure comprising:
a base portion;
a pressure equalization sheet placed on one side of the base portion and filled with liquid or gas;
a sensor unit including at least one sensor placed on one side of the pressure equalization sheet; and
stoppers configured to be in close contact with the pressure equalization sheet and face each other.

2. The sensor module of claim 1, wherein
heights of the stoppers are lower than the pressure equalization sheet.

3. The sensor module of claim 1, wherein
each of the stoppers has one of a bar shape and an arc shape.

4. The sensor module of claim 1, wherein
the stoppers surround the pressure equalization sheet.

5. The sensor module of claim 1, further comprising:
a circuit,
wherein the circuit includes:
a communication unit configured to communicate with an external device;
a controller configured to receive a sensor signal measured by the sensor unit;
a display configured to display information received from the controller; and
a battery configured to supply power to at least one of the communication unit, the controller, the display, and the sensor unit.

6. The sensor module of claim 1, wherein
the sensor unit includes:
a first sensor;
a second sensor separated by a preset distance from the first sensor; and
a step difference portion placed on one side of the first sensor, and
a reference surface of the first sensor has a step difference by a first distance with a reference surface of the second sensor.

7. The sensor module of claim 6, wherein
the sensor unit further includes a third sensor separated by a preset distance from the second sensor.
